(19) United States
(12) Patent Application Publication
(10) Pub. No.: US 2025/0361457 A1
(43) Pub. Date: Nov. 27, 2025

(54) DISSOLVED C60 AND METHOD OF PRODUCING DISSOLVED C60

(71) Applicant: Innovation for Success, LLC, Doylestown, PA (US)

(72) Inventor: Leonard W. Heflich, Doylestown, PA (US)

(21) Appl. No.: 19/293,395

(22) Filed: Aug. 7, 2025

Related U.S. Application Data
(63) Continuation-in-part of application No. 17/809,179, filed on Jun. 27, 2022.
Application No. 17/809,179 is a continuation-in-part of application No. 17/078,554, filed on Oct. 23, 2020, now Pat. No. 11,400,113.
Application No. 17/078,554 is a continuation-in-part of application No. 16/819,552, filed on Mar. 16, 2020, now Pat. No. 10,842,742.
(60) Provisional application No. 62/884,198, filed on Aug. 8, 2019.

(51) Int. Cl. C10M 125/02; C10M 105/04; C10M 129/06; C10N 40/25; C10N 40/30; C10N 50/00; C10N 50/10; C10N 70/00

(52) U.S. Cl. CPC C10M 125/02; C10M 105/04; C10M 129/06; C10M 2201/041; C10M 2203/0206; C10M 2207/021; C10N 2040/25; C10N 2040/30; C10N 2050/01; C10N 2050/10; C10N 2070/00

(57) ABSTRACT

A method of dispersing C60 in a lubricant to form a C60 lubricant includes combining C60 with a limonene composition to form a C60 mixture, and heating the C60 mixture to a predetermined temperature for a predetermined time period to dissolve the C60 into the limonene composition to form a C60 concentrate. The method further includes combining the C60 concentrate with a cosolvent to form a blended C60 mixture, and combining the blended C60 mixture with the lubricant to disperse the C60 in the lubricant and to form the C60 lubricant.

This application is a continuation-in-part of U.S. application Ser. No. 17/809,179, filed on Jun. 27, 2022, which is a continuation-in-part of U.S. application Ser. No. 17/078,554, filed on Oct. 23, 2020 (now U.S. Pat. No. 11,400,113, issued Aug. 2, 2022), which is a continuation-in-part of U.S. application Ser. No. 16/819,552, filed on Mar. 16, 2020 (now U.S. Pat. No. 10,842,742, issued Nov. 24, 2020), which claims the benefit of priority of U.S. provisional application Ser. No. 62/884,198, filed on Aug. 8, 2019, the disclosures of which are herein incorporated by reference in their entirety.

### Field

This disclosure relates to the field of nanomaterials and, in particular, to dissolving nanomaterials in a liquid or semi-solid lubricant, such as motor oil or grease. The resultant C60 lubricant product has improved tribological properties and is suitable for lubricating high-precision moving components, moving parts under load such as in a gearbox, reducing wear, and reducing friction.

### Background

Nanomaterials are materials of which a single unit is sized, in at least one dimension, from approximately one nanometer (1 nm) to approximately one thousand nanometers (1,000 nm) and often from approximately one nanometer (1 nm) to approximately one hundred nanometers (100 nm). One type of nanomaterial or sub-nanomaterial is the C60 molecule, which is also referred to as C.60, C-60, C₆₀, Buckminsterfullerene, fullerene, and buckyballs. The C60 molecule is an allotrope of carbon and consists of carbon atoms connected by single and double bonds so as to form a closed mesh. Each C60 molecule includes sixty atoms of carbon arranged in a soccer ball-like shape (see FIG. 1) that includes twenty hexagons and twelve pentagons with a carbon atom at each vertex of each hexagon and pentagon. The C60 molecules have a diameter of approximately 0.72 nm; thus, C60 is typically referred to as either a nanomaterial or a sub-nanomaterial.

C60 is typically formed through a combustion process that isolates C60 molecules from soot. To prepare C60 for tribological usage, it would be desirable to mix C60 with a lubricant, such as oil, grease, or another lubricating material. However, known processes for dissolving and dispersing C60 in oil or grease are extremely time consuming and expensive because C60 dissolves only to a trivial extent in oil and grease. Moreover, C60 tends to agglomerate when mixed with oil and other lubricants. The agglomerates of C60 are very hard and very sharp, and are capable of damaging metal surfaces. For example, when C60 powder is added directly to the motor oil of an internal combustion engine, the resulting agglomerates are likely to damage the cylinder walls and the main bearings during operation of the engine. As such, directly adding powdered C60 or crystallized C60 to a liquid lubricant, such as motor oil, or a semi-solid lubricant, such as grease, is not a feasible approach for improving the lubricity and lubricating performance of the lubricant.

To attempt to overcome this issue, researchers add C60 powder directly to oil and then sonicate the C60 and oil mixture for five to ten days to break up the agglomerates. Even after this extremely lengthy and inefficient process, however, only a trivial amount of the C60 becomes dissolved in the oil and significant numbers of agglomerates remain, with some remaining agglomerates being large enough to damage high-precision equipment and to score highly-polished surfaces. Thus, according to known processes and methods, combinations of C60 and oil are difficult and time consuming to prepare for tribological applications.

Based on the above, further developments in the area of preparing C60 as an additive to liquid and semi-solid lubricants for tribological applications are desired.

### Summary

According to an exemplary embodiment of the disclosure, a method of dispersing C60 in a lubricant to form a C60 lubricant includes combining C60 with a limonene composition to form a C60 mixture, and heating the C60 mixture to a predetermined temperature for a predetermined time period to dissolve the C60 into the limonene composition to form a C60 concentrate. The method also includes combining the C60 concentrate with a cosolvent to form a blended C60 mixture, and combining the blended C60 mixture with the lubricant to disperse the C60 in the lubricant and to form the C60 lubricant.

According to a further exemplary embodiment of the disclosure, a method of dispersing C60 powder in a liquid lubricant or semi-solid lubricant to form a C60 lubricant includes dissolving the C60 powder in limonene to form a C60 concentrate by heating the C60 powder and limonene to a predetermined temperature for a predetermined time period to dissolve the C60 powder into the limonene to fully dissolve the C60 powder. The method further requires combining the C60 concentrate with a cosolvent to form a blended C60 mixture, and combining the blended C60 mixture with the liquid lubricant or semi-solid lubricant to disperse the C60 in the lubricant and to form a uniform C60 lubricant having fully dispersed and stable C60. The cosolvent enables the C60 concentrate to be dispersed into the liquid lubricant or the semi-solid

According to another exemplary embodiment of the disclosure, a method of forming a C60 lubricant includes forming a C60 mixture by combining C60 powder with a limonene composition, and heating the C60 mixture to a predetermined temperature for a predetermined time period to dissolve the C60 powder into the limonene composition to form a C60 concentrate. The method further includes forming a blended C60 mixture by combining the C60 concentrate with a cosolvent, and forming the C60 lubricant by combining the blended C60 mixture with an oil.

### BRIEF DESCRIPTION OF THE FIGURES

FIG. 1 is a block diagram of a C60 molecule;

FIG. 2 is a diagram showing the constituent parts involved in the formation of a C60 lubricant;

FIG. 3 is a flowchart illustrating an exemplary method of forming the C60 lubricant;

FIG. 4 is a plan view at 1000× magnification showing C60 combined with a limonene composition to form a C60 mixture at the beginning of the method of FIG. 3;

FIG. 5 is another plan view at 1000× magnification showing the C60 mixture twenty minutes into the method of FIG. 3;

FIG. 6 is a further plan view at 1000× magnification showing the C60 mixture twenty-five minutes into the method of FIG. 3; and

FIG. 7 is a plan view at 1000× magnification showing that the C60 mixture has turned into the C60 concentrate thirty minutes into the method of FIG. 3.

### DETAILED DESCRIPTION

For the purpose of promoting an understanding of the principles of the disclosure, reference will now be made to the embodiments illustrated in the drawings and described in the following written specification. It is understood that no limitation to the scope of the disclosure is thereby intended. It is further understood that this disclosure includes any alterations and modifications to the illustrated embodiments and includes further applications of the principles of the disclosure as would normally occur to one skilled in the art to which this disclosure pertains.

Aspects of the disclosure are disclosed in the accompanying description. Alternate embodiments of the disclosure and their equivalents may be devised without parting from the spirit or scope of the disclosure. It should be noted that any discussion herein regarding “one embodiment,” “an embodiment,” “an exemplary embodiment,” and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

For the purposes of the disclosure, the phrase “A and/or B” means (A), (B), or (A and B). For the purposes of the disclosure, the phrase “A, B, and/or C” means (A), (B), (C), (A and B), (A and C), (B and C), or (A, B and C).

The terms “comprising,” “including,” “having,” and the like, as used with respect to embodiments of the disclosure, are synonymous.

As used herein, the term “approximately” means within plus or minus 5% of the stated value.

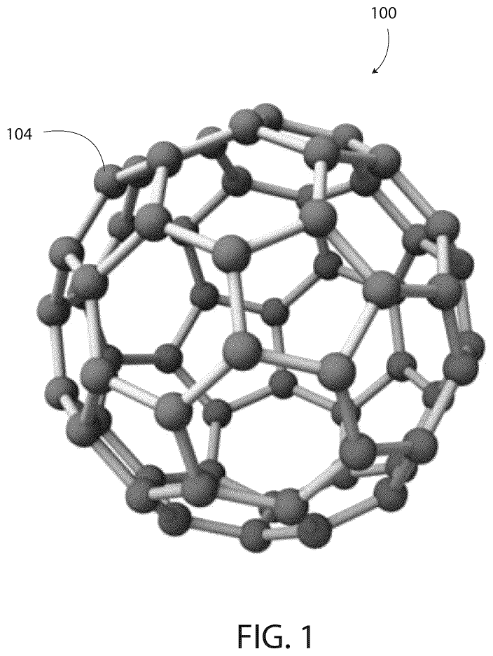

As shown in FIG. 1, a C60 molecule 100 includes sixty atoms of carbon 104 arranged in a soccer ball-like shape. C60 molecules 100 are available as a powder that is formed by pulverizing masses or crystals of the C60 molecules 100. As used herein, the term “C60” refers to the C60 molecules 100 in the powered form (C60 powder), the crystalized form, the dissolved form, and in any other form.

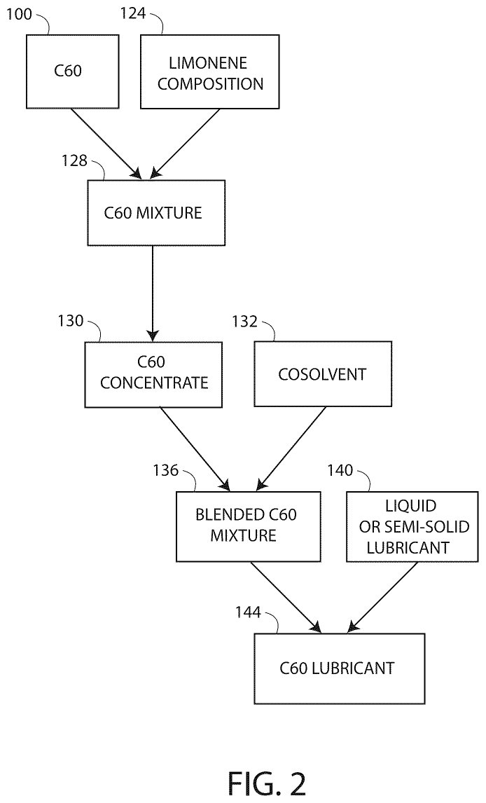

With reference to FIG. 2, when C60 100 is mixed with a corresponding lubricant 140, according to the inventive approach disclosed herein, a resultant C60 lubricant 144 is formed that has improved tribological properties. For example, the C60 lubricant 144 reduces friction between two moving elements and reduces wear between two moving elements, as compared to the lubricant 140 alone. The hard and generally-spherical molecular shape of the C60 100 is instrumental to the improved lubricating properties of the C60 lubricant 144. As described herein, the lubricant 140 includes, but is not limited to, liquid lubricants, such as oil, and semi-solid lubricants, such as grease.

As mentioned above, however, an obstacle to preparing C60 100 for tribological purposes is that when C60 100 is added directly to the lubricant 140, the C60 100 will not dissolve and/or disperse well (or at all), and the C60 100 tends to clump together in masses (i.e., agglomerates). Table 1, included herein, identifies the solubility of C60 100 in various liquids. As shown, C60 100 dissolves in oil (an exemplary liquid lubricant 140) only to a trivial extent (olive oil (0.9 mg/ml)). As a result, when added directly to the lubricant 140, an insignificant amount of the C60 100 becomes dissolved and/or dispersed in the lubricant 140 and the tribological properties of the lubricant 140 are either unchanged or are reduced due to the presence of the agglomerates of C60 100.

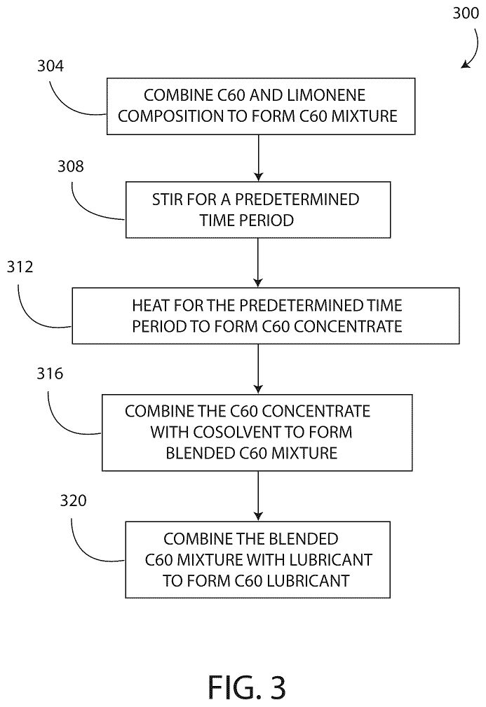

With continued reference to FIG. 2, in an unexpected breakthrough, a cosolvent approach is used to quickly and easily dissolve, disperse, and/or emulsify the C60 100 in the liquid or semi-solid lubricant 140 for tribological applications. According to the cosolvent approach, first the C60 100 is mixed with a limonene composition 124 to form a C60 mixture 128. The C60 mixture 128 is heated to form a C60 concentrate 130. The C60 concentrate 130 is homogenous and stable, with free C60 molecules 100 dissolved in the limonene composition 124. Then, the C60 concentrate 130 is combined with a cosolvent 132 to form a blended C60 mixture 136. Next, the blended C60 mixture 136 is added to the liquid or semi-solid lubricant 140, according to a mixing approach, to form the C60 lubricant 144. The result of this inventive method 300 (FIG. 3) is a stable and homogenous solution of dispersed, dissolved, and/or emulsified C60 100 in the lubricant 140. The C60 lubricant 144 has improved tribological properties as compared to the lubricant 140 alone. Each step of the method 300 is described in detail herein.

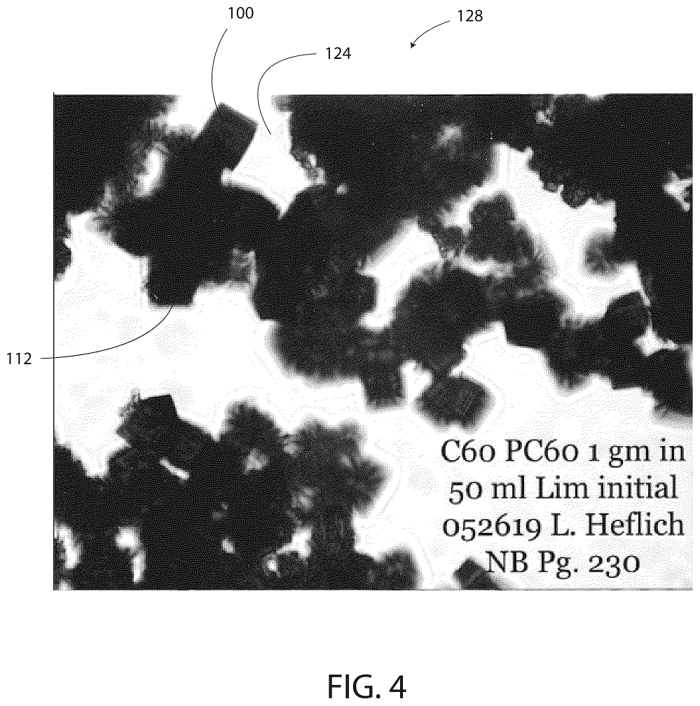

As shown in the flowchart of FIG. 3 and with additional reference to FIGS. 4-7, the method 300 is for efficiently dispersing, dissolving, and/or emulsifying the C60 100 in the lubricant 140 to form the C60 lubricant 144. In block 304, the method 300 includes combining the C60 100 with the limonene composition 124 to form the C60 mixture 128. Depending on the embodiment, the limonene composition 124 is 100% limonene, 100% d-limonene, or mixtures of limonene and d-limonene in any percentage by volume or weight. In one embodiment, the limonene composition 124 is at least 95% d-limonene by volume, with the remaining 5% including any other liquid, such as limonene, flavoring, fragrances, and the like. In another embodiment, the limonene composition 124 is at least 75% d-limonene by volume with the remaining 25% including any other liquid, such as limonene, other essential oils, flavoring, fragrances, and/or other monoterpenes and di-terpenes such as alpha-pinene, beta-caryophyllene, gamma terpinene, and linalool.

Limonene and d-limonene are excellent liquid media for dissolving the C60 100. Limonene is a colorless and transparent liquid aliphatic hydrocarbon classified as a cyclic terpene, and is a major component in the oil of citrus fruit peels. Limonene is a chiral molecule. D-limonene is the d-isomer of limonene and has a strong smell of oranges and a bitter taste. D-limonene is used as a fragrance ingredient in cosmetic products and also as a flavoring agent in food manufacturing. D-limonene, which is a monoterpene, is obtained commercially from citrus fruits through centrifugal separation or steam distillation, for example. D-limonene is a colorless and transparent liquid. D-limonene is plentiful and inexpensive.

At block 304 of the method 300, the C60 100, which, in one embodiment, has been pulverized into a powdered form (C60 powder), is combined with the liquid limonene composition 124 in a glass vessel (not shown) or any other heat-safe and non-reactive container to form the C60 mixture 128. Additionally or alternatively, C60 100 that is not pulverized is added to the limonene composition 124 in granular, chunky, or crystalline state to form the C60 mixture 128.

In FIG. 4, the C60 mixture 128 illustrated at room temperature (also referred to herein as an ambient temperature) of about 70° F. (21° C.) shortly after the C60 100 is added to the limonene composition 124. FIG. 4 is a 1000× magnification view of a portion of one gram (1 g) of pulverized C60 100 mixed with sixty milliliters (60 ml) of the limonene composition 124.

The C60 100 has formed small clumps 112 (agglomerates) and has not immediately dissolved into the limonene composition 124. When viewed in color, at this stage of the method 300 (block 304), the C60 mixture 128 is colorless and transparent, and the C60 100 has a mostly black color. Any apparent color of the C60 mixture 128 in FIG. 4 is the result of an incandescent light source emitting yellowish hue in certain photographic representations.

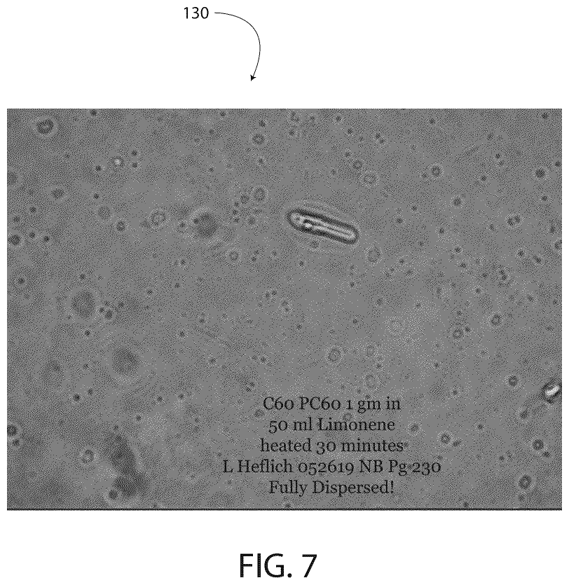

Next, at blocks 308 and 312 of the method 300, the C60 mixture 128 is stirred and heated for a predetermined time period to form the C60 concentrate 130 (FIGS. 2 and 7). An exemplary predetermined time period is from twenty to forty minutes. In one embodiment, the predetermined time period is thirty minutes or approximately thirty minutes.

In one example, the C60 mixture 128 stirred with a magnetic stirring system (not shown) that uses a rotating magnetic field to cause a stir bar immersed in the C60 mixture 128 to move. In other embodiments, any other stirring system may be utilized to stir the C60 mixture 128 including hand stirring with a suitable tool, such a glass stirrer shaft (not shown). Moreover, in other embodiments, no stirring of the C60 mixture 128 is performed and only the heating process (block 312) is used to dissolve the C60 100 (C60 powder) into the limonene composition 124. The stirring step of block 308 is an optional part of the method 300.

At block 312, the C60 mixture 128 is heated during the predetermined time period to a predetermined temperature by a hot plate or any other electric heating element system. An exemplary predetermined temperature is from approximately 250° F. to approximately 300° F. (121° C. to 149° C.). In one embodiment, the predetermined temperature is 275° F. (135° C.) or approximately 275° F. (135° C.). Optionally, the C60 mixture 128 is covered during the heating process (block 312), such as with a watch glass or any other suitable cover, to facilitate solvent reflux, returning the condensed solvent (i.e. the limonene composition 124) to the body of the solution. Very little of the limonene composition 124 evaporates during the method 300. In one embodiment, convection currents of the heated C60 mixture 128 provide movement of the liquid to result in dissolving of the C60 100 without the stirring step of block 308.

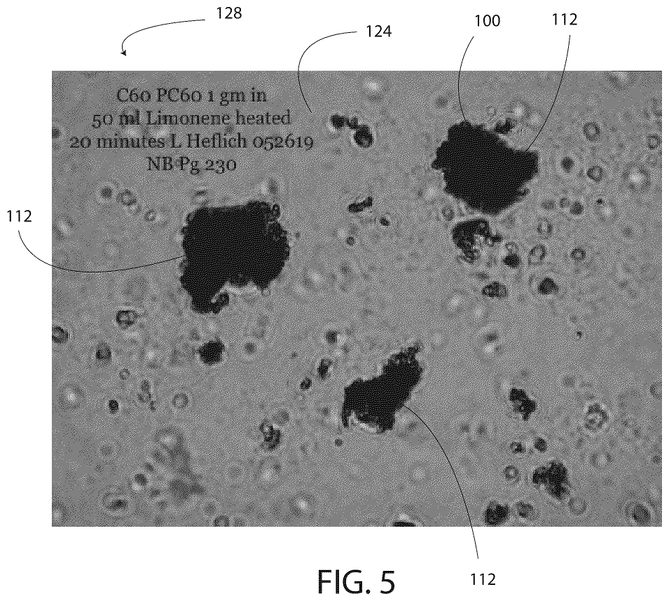

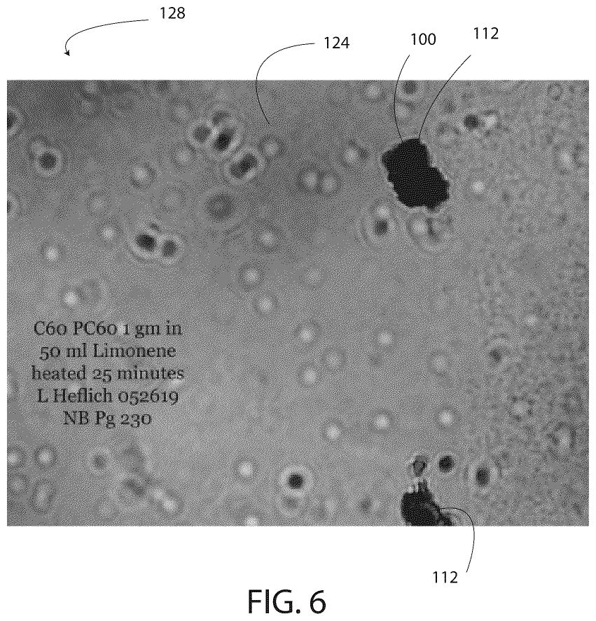

The stirring and heating of blocks 308 and 312 dissolves the C60 100 (C60 powder) into the limonene composition 124. FIGS. 5 and 6 illustrate two additional stages of the stirring and heating process of the C60 mixture 128. In FIG. 5, the C60 mixture 128 has been heated for twenty minutes and fewer clumps 112 of the C60 100 remain, as compared to FIG. 4, thereby indicating that more of the C60 100 has dissolved into the limonene composition 124. When viewed in color, the C60 mixture 128 has become orange at the process stage illustrated in FIG. 5 (i.e., block 312).

In FIG. 6, the C60 mixture 128 has been heated for twenty-five minutes and even fewer of the clumps 112 of the C60 100 remain, as compared to FIG. 5. The remaining clumps 112 of the C60 100 are very small. When viewed in color, the C60 mixture 128 has an orange color at the process stage illustrated in FIG. 6 (i.e., block 312).

With reference to FIG. 7, the C60 mixture 128 has been heated for thirty minutes and no clumps 112 of the C60 100 remain, indicating that the C60 concentrate 130 is formed. The C60 concentrate 130 is a homogenous liquid including C60 molecules 100 that are fully dissolved into the limonene composition 124 with no clumps 112 of the C60 100. The C60 concentrate 130 is a clear solution (i.e. transparent) with a deep magenta color. Any artifacts illustrated in FIG. 7 are air bubbles or other features, and are not the clumps 112 of the C60 100.

In some embodiments, at blocks 308 and 312 of the method 300, the C60 mixture 128 is sonicated to reduce the time required to dissolve the C60 100 into the limonene composition 124 and to assist in breaking up any of the clumps 112 of the C60 100. Sonication is a process of applying sound energy to the C60 mixture 128, thereby agitating the clumps 112 of the C60 100 and promoting full and timely dissolution of the C60 molecules 100 into the limonene composition 124. A sonication system (not shown) may be placed near or in the C60 mixture 128 to perform the sonication. The sonication, in one embodiment, is strong enough to cause cavitation within the C60 mixture 128. During the cavitation microscopic bubbles are formed and collapse thereby promoting the dissolution of the C60 100 into the limonene composition 124.

As used herein, “dissolving” the C60 100 into the limonene composition 124 refers to forming a solution including a homogenous mixture of C60 molecules 100 and molecules of the limonene composition 124. In such a solution, the C60 100 is the solute and the limonene composition 124 is the solvent. In some embodiments, atomic level changes and bonds may occur between the dissolved C60 100 and molecules of the limonene composition 124; however, the C60 molecules 100 retain their shape as shown in FIG. 1 as well as their tribological properties. The C60 concentrate 130 may also be referred herein to as a dissolved C60 mixture and/or a C60 solution including solubilized C60 100 in the limonene composition 124.

In an exemplary embodiment, the C60 concentrate 130 includes at least 16.67 mg of dissolved C60 100 per one milliliter (1 ml) of the limonene composition 124. At the solubility of 16.67 mg/ml, the C60 concentrate 130 is “stable,” meaning that no settling or precipitating of the C60 100 occurs in the limonene composition 124, even after six weeks of sitting at room temperature. As disclosed herein, a limit of solubility of the C60 100 in the limonene composition 124 is from approximately 16.67 mg/ml to approximately 20.0 mg/ml. Accordingly, in other embodiments, the C60 concentrate 130 includes from 0.50 mg to 20.0 mg of the dissolved C60 100 per one milliliter (1 ml) of the limonene composition 124.

When the C60 concentrate 130 is made according to the method 300 of FIG. 3 with more than 16.67 mg of C60 100 per milliliter of the limonene composition 124, some settling of the C60 100 occurs when the C60 concentrate 130 is cooled from the predetermined temperature to room temperature. The settled C60 100, however, can be easily “redissolved” by gently shaking and/or agitating the C60 concentrate 130 at room temperature (i.e. without reheating). Whereas, when the C60 concentrate 130 is made according to the method 300 of FIG. 3 with 16.67 mg or less of the C60 100 per milliliter of the limonene composition 124, no settling of the C60 100 occurs when the C60 concentrate 130 is cooled from the predetermined temperature to room temperature, and the C60 concentrate 130 is stable. For a point of reference, the limit of solubility of the C60 100 in toluene is 2.8 mg/ml and the limit of solubility of the C60 100 in olive oil is 0.9 mg/ml. Thus, the method 300 produces a solution with a high concentration of dissolved C60 molecules 100 per milliliter of solvent.

The method 300 of FIG. 3 works to dissolve all of the commercially available forms of C60 100 including those types of C60 100 having a snowflake appearance under magnification and those types of C60 100 having a crystalline (i.e. cubic or hexagonal) appearance under magnification.

As noted above, the stirring process of block 308 is optional, but the heating process of block 312 is typically performed. As disclosed herein, the C60 100 typically does not dissolve to any significant extent into the limonene composition 124 when the C60 mixture 128 is at room temperature 70° F. (21° C.). However, when the C60 mixture 128 is heated to the predetermined temperature, dissolution of the C60 100 into the limonene composition 124 occurs.

Next, at block 316 of the method 300, the C60 concentrate 130 is combined with a cosolvent 132 to form a blended C60 mixture 136 that includes the cosolvent 132, the limonene composition 124, and the dissolved C60 100. The cosolvent 132 is a secondary solvent that is used to improve the solubility of the C60 concentrate 130 in the lubricant 140. The cosolvent 132 is used because the C60 concentrate 130 is not efficiently dispersed directly into the lubricant 140. Thus, the cosolvent 132, as described below, enables the dispersion and combination of the C60 100 with the lubricant 140. Specifically, when the C60 concentrate 130 is added directly to the lubricant 140 (provided as oil, in one embodiment), the C60 concentrate 130 settles to the bottom of the lubricant 140 and forms an intractable viscous fluid. The viscous fluid remains and/or returns even after mixing using sonication and/or high sheer mixing. The cosolvent 132 completely prevents the formation of the viscous fluid and enables the C60 concentrate 130 to evenly disburse into the lubricant 140.

In one embodiment, the cosolvent 132 is miscible in limonene and d-limonene and is moderately polar. As used herein, a moderately polar cosolvent 132 has a normalized empirical polarity parameter (ETN or ETN) from 0.3 to 0.7, and preferably from 0.5 to 0.7. An exemplary cosolvent 132 is or includes benzyl alcohol. Benzyl alcohol is a preferred cosolvent 132 because it has an ETN of 0.6 and a boiling point of 410° F. (210° C.), which is well above the typical operating point of an internal combustion engine, transmission, gearbox, or a refrigerant compressor of a refrigeration system, which are exemplary uses for the C60 lubricant 144.

At block 316 of the method 300, the C60 concentrate 130 is mixed with the cosolvent 132 at a predetermined ratio to form the blended C60 mixture 136. For example, in an embodiment, the C60 concentrate 130 and the cosolvent 132 are mixed at a 1:1 ratio by volume or a 2:1 ratio by volume to form the blended C60 mixture 136. In other embodiments, the C60 concentrate 130 and the cosolvent 132 are mixed a ratio from 1:2 to 10:1 to form the blended C60 mixture 136, where the first digit in the ratio is the amount of the C60 concentrate 130 by volume and the second digit in the ratio is the amount of the cosolvent 132 by volume. Thus, the 2:1 ratio includes two parts of the C60 concentrate 130 and one part of the cosolvent 132.

In an embodiment of the method 300, at block 316, the blended C60 mixture 136 is formed with both the C60 concentrate 130 and the cosolvent 132 being at room temperature when combined. This approach, therefore, includes cooling the C60 concentrate 130 to room temperature or to near room temperature after the heating of block 312.

Next, at block 320 of the method 300, the blended C60 mixture 136 is combined with the lubricant 140 to disperse the C60 100 into and throughout the lubricant 140, thereby forming the C60 lubricant 144. The lubricant 140, in one embodiment, is provided as a liquid lubricant including, but not limited to, engine oil (motor oil) for an internal combustion engine, gear oil for a corresponding gear box, and transmission fluid for a corresponding transmission. Accordingly, the lubricant 140 is provided as a liquid petroleum-based motor oil, a partially-synthetic motor oil, or a fully-synthetic motor oil. The lubricant 140, in another embodiment, is provided as a semi-solid lubricant including, but not limited to, grease. At block 320, the blended C60 mixture 136 is combined with any liquid lubricant or semi-solid lubricant 140 to form the C60 lubricant 144.

When the lubricant 140 is an engine oil, the C60 lubricant 144 has improved lubricating effects as compared to the engine oil alone when used in the internal combustion engine. That is, the C60 lubricant 144 reduces the friction between the piston rings and the cylinder walls and reduces the friction between the crankshaft and the main bearings.

In another embodiment, the lubricant 140 is a liquid lubricating oil for lubricating a corresponding refrigerant compressor of a refrigerant system and, therefore, the resultant C60 lubricant 144 is compatible with the refrigerant of the refrigerant system. The mentioned “compatibility” means that the C60 lubricant 144 does not chemically react with the refrigerant to form a sludge, acid, or other harmful byproduct that could damage or reduce the effectiveness of a component of the refrigeration system. The compressor compresses the refrigerant, and the C60 lubricant 144 reduces wear and friction of the operating compressor more than the lubricant 140 alone.

In a further embodiment, the C60 lubricant 144 is formed by blending C60 mixture 136 directly with a semi-solid lubricant 140, such as grease. The resultant C60 lubricant 144 is used in gearboxes and bearings to reduce friction, improve mechanical efficiency and reduce wear, thereby extending service life. In such an application, the C60 lubricant 144 formed as a combination of the blended C60 mixture 136 and the semi-solid lubricant 140, has improved lubricating properties as compared to the grease alone. For example, according to the American Society for Testing and Materials test D4172 (ASTM D4172, “Standard Test Method for Wear Preventive Characteristics of Lubricating Fluid (Four-Ball Method)”), the C60 lubricant 144 formed as a combination of the blended C60 mixture 136 and the semi-solid lubricant 140 (i.e., grease) resulted in a friction reduction of 17% and a wear reduction of 12.6% as compared to the grease alone.

At block 320 of the method 300, in one embodiment, the lubricant 144 is sonicated as the blended C60 mixture 136 is added to the lubricant 144. Sonicating the lubricant 144 includes applying sound energy to the lubricant 144, thereby agitating the lubricant 144 and the blended C60 mixture 136, and promoting full and timely dissolution and/or dispersion and/or emulsification of the blended C60 mixture 136 into the lubricant 140. The sonication, in one embodiment, is strong enough to cause cavitation within the lubricant 140. During the cavitation, microscopic bubbles are formed and collapse thereby promoting the dispersion of the blended C60 mixture 136 into the lubricant 144. The sonication of the lubricant 144 and the blended C60 mixture 136 is performed from thirty to three hundred seconds, and preferably for one hundred seconds. The sonication process disperses, emulsifies, and/or homogenizes the blended C60 mixture 136 with the lubricant 140. Thus, in at least one embodiment, the C60 lubricant 144 is a stable emulsion of the blended C60 mixture 136 and the lubricant 140.

Additionally or alternatively at block 320 of the method 300, the lubricant 140 is mixed with a high shear mixer as the blended C60 mixture 136 is added to the lubricant 140. The high shear mixer includes a rotor that spins from 7,000 rotations per minute to 10,000 rotations per minute during the high shear mixing process. The high sheer mixing process disperses, emulsifies, and/or homogenizes the blended C60 mixture 136 with the lubricant 140. The high sheer mixing approach, in one embodiment, also results in the C60 lubricant 144 that is a stable emulsion of the blended C60 mixture 136 and the lubricant 140.

At block 320, according to one embodiment, the lubricant 140 and the blended C60 mixture 136 are combined with both the lubricant 140 and the blended C60 mixture 136 being at or near room temperature. Accordingly, the method 300 does not require heating of either the lubricant 140 or the blended C60 mixture 136 in order to form the C60 lubricant 144. In other embodiments and depending on at least the characteristics of the lubricant 140, during the combining at block 320 at least one of the lubricant 140 and the blended C60 mixture 136 is heated. For example, heating the lubricant 140 as the blended C60 mixture 136 is added may reduce the duration of sonication and/or high sheer mixing that is required to form the C60 lubricant 144. Such a heating approach is optionally used when the lubricant 140 is a semi-solid, such as grease.

Due to the presence of the cosolvent 132 when the blended C60 mixture 136 is combined with the lubricant 140, the blended C60 mixture 136 evenly disperses through the lubricant 140, thereby resulting in the C60 molecules 100 also being evenly dispersed through the lubricant 140. When added to the lubricant 140 the blended C60 mixture 136 does not settle to the bottom of the lubricant 140 and does not form the previously-described intractable viscous fluid. Instead, the blended C60 mixture 136 immediately begins to dissolve and disperse into the lubricant 140 to form the C60 lubricant 144. This is a breakthrough technique that enables the efficient dispersion of the C60 100 into and throughout the lubricant 140.

The C60 lubricant 144 is stable, such that no settling or precipitating of the C60 100 occurs. That is, the C60 100 does not separate and settle to the bottom of a container of the C60 lubricant 144, even when the C60 lubricant 144 is left undisturbed for weeks at a time. Moreover, no settling of the C60 mixture 128, the C60 concentrate 130, and the blended C60 mixture 136 occurs within the lubricant 140. Instead, in one embodiment, depending at least on the composition of the cosolvent 132 and the lubricant 140, the resultant C60 mixture 128 is an emulsion of the lubricant 140 and the blended C60 mixture 136, in which the C60 100 is evenly and permanently dispersed throughout the lubricant 140. According to some embodiments, the cosolvent 132 is an emulsifier or surfactant that stabilizes the emulsion. The C60 lubricant 144, in some embodiments, remains a very fine and stable emulsion even after being heated to over 330° F. (166° C.).

The C60 lubricant 144 is made with the C60 concentrate 130. Accordingly, there are no agglomerates or clumps 112 in the C60 lubricant 144. Instead, the C60 100 in the C60 lubricant 144 is completely dissolved. This makes the C60 lubricant 144 particularly well-suited for high-precision applications that would be damaged by the clumps 112. For example and depending on the characteristics of the selected lubricant 140, as mentioned above, the C60 lubricant 144 is well-suited for internal combustion engine applications and refrigerant compressor applications. The C60 lubricant 144 has an improved lubricity over the lubricant 140 alone. Therefore, the C60 lubricant 144 improves the fictional and wear characteristics over any corresponding lubricant 140 alone.

In use, the C60 lubricant 144 lubricates components with both the lubricant 140 and the C60 100. Due to the shape of the C60 molecules 100, the C60 100 within the C60 lubricant 144 function as microscopic ball bearings. The microscopic ball bearings are suspended freely in the lubricant 140. The C60 molecules 100 are very strong and resilient, such that no damage or breakdown of the C60 100 occurs, even in high pressure and/or high temperature conditions. The C60 100 is chemically inert so no reaction occurs even after many hours of operation in an internal combustion engine, for example.

The method 300 of forming the C60 lubricant 144 is orders of magnitude faster than the known process of mixing C60 100 in olive oil. Mixing C60 100 in olive oil takes approximately seven days in order to somewhat break up the clumps of C60 100, and over the course of the seven days, only a trivial amount of the C60 100 is dissolved in the olive oil with the rest of the C60 100 being unevenly dispersed through the olive oil in small (e.g. microscopic) clumps. The method 300, as disclosed herein, completely dissolves the C60 100 in only thirty minutes by forming the C60 concentrate 130, then by using the cosolvent 132, the blended C60 mixture 136 is combined with the lubricant 140 in minutes (from one to thirty minutes) depending on the quantity and the mixing capabilities. As a result, the method 300 offers huge time savings in the preparation of a stable C60 lubricant 144 and provides a C60 lubricant 144 having no agglomerate or clumps 112 that could potentially damage high-precision equipment.

The C60 lubricant 144 has a predetermined amount of C60 100 per unit volume. For example, in one embodiment using the limonene composition 124 and benzyl alcohol as the cosolvent 132, the C60 lubricant 144 includes 0.835 mg of the C60 100 in each one milliliter of the C60 lubricant 144 (i.e., 0.835 mg/ml). The solubility of the C60 in olive oil is 0.9 mg/ml; thus, the method 300 achieves 92.8% of saturation (92.8% of the solubility limit) in the exemplary lubricant 140 provided as motor oil or compressor oil.

Testing has also been performed according to ASTM D4172 to confirm the tribological benefits of the C60 lubricant 144 when the lubricant 140 is provided as a liquid oil. For example, the lubricant 140 was provided as Pennzoil Platinum Full Synthetic SAE OW-20 motor oil. The grand average coefficient of friction was 0.097 for the control (the Pennzoil alone) and 0.080 for the C60 lubricant 144. Thus, the C60 lubricant 144 exhibited a 17.5% reduction in overall coefficient of friction. Concerning the ball wear, the control measured 0.46 mm of surface wear and the C60 lubricant 144 exhibited 0.37 mm of surface wear. Thus, the C60 lubricant 144 exhibited a 19.6% reduction in wear under the test conditions (40 kg at 1200 RPM for one hour at 167° F., 75° C.).

TABLE 1
Solubility of C60 in Various Solvents

| Solvent | Solubility Limit in mg/ml | Edible |
|---|---|---|
| methanol | ND | No |
| tetrahydrofuran | ND | No |
| isopropanol | ND | No |
| water | 1.3 × 10⁻¹¹ | Yes |
| acetone | 0.001 | No |
| ethanol | 0.0014 | Yes |
| n-pentane | 0.005 | No |
| cyclohexane | 0.036 | No |
| octanol | 0.0429 | No |
| n-hexane | 0.043 | No |
| n-decane | 0.071 | No |
| chloroform | 0.16 | No |
| dichloromethane | 0.26 | No |
| tetrachloromethane | 0.32 | No |
| benzonitrile | 0.41 | No |
| carbon tetrachloride | 0.447 | No |
| olive oil | 0.9 | Yes |
| benzene | 1.7 | No |
| toluene | 2.8 | No |
| decalins | 4.6 | No |
| xylene | 5.2 | No |
| anisole | 5.6 | No |
| carbon disulfide | 7.9 | No |
| tetralin | 16 | No |
| d-limonene | 16.67 | Yes |
| 1,2-dichlorobenzene | 27 | No |
| 1-methylnaphthalene | 33 | No |
| 1-chloronaphthalene | 41 | No |

As disclosed herein, according to a further approach, the C60 lubricant 144 is used to form a lubricating C60 grease. This is an additional and different approach than combining the blended C60 mixture 136 with the lubricant 140 provided as grease. In this different approach, the blended C60 mixture 136 is combined with the lubricant 140 provided as a liquid, such as oil. Then, the C60 grease is formed by combining the resultant C60 lubricant 144 with a thickener. The C60 grease retains the semi-solid consistency of the original grease.

Exemplary thickeners include soap-based thickeners, such as lithium, calcium, and aluminum. Other exemplary thickeners include non-soap thickeners, such as polyurea, clay, and silica.

According to another approach, the C60 100 is added to gasoline, to improve the operation of the corresponding internal combustion engine. In this approach, the C60 100 is dissolved in toluene to form a C60 toluene mixture. As shown in table 1, C60 100 is soluble in toluene. The C60 toluene mixture is then added to the gasoline. Gasoline is already from 5% to 35% toluene by volume depending on the formulation. Thus, the C60 toluene mixture is an efficient approach for dispersing C60 into gasoline. In operation, the C60 100 enters the engine through the engine intake system, and then the C60 100 is available to disperse into the lubricating oil of the engine as a result of the combustion process. The C60 100 in the gasoline also lubricates fuel injectors and valve systems as the C60 100 moves through the internal combustion engine.

The C60 toluene mixture is typically not used to form the C60 lubricant 144. That is, toluene is not used at the cosolvent 132. This is because when toluene is added to the lubricant 140, the toluene changes the viscosity and degrades the lubricity of the lubricant 140. In addition, the low boiling point of toluene (231.1° F., 110.6° C.) results in evaporation of the toluene in an operating internal combustion engine and degradation of the corresponding emulsion that is the C60 lubricant 144. Moreover, C60 100 has a much lower solubility in toluene (2.8 mg/ml) as compared to d-limonene (16.67 mg/ml).

While the disclosure has been illustrated and described in detail in the drawings and foregoing description, the same should be considered as illustrative and not restrictive in character. It is understood that only the preferred embodiments have been presented and that all changes. modifications and further applications that come within the spirit of the disclosure are desired to be protected.

What is claimed is:

1. A method of dispersing C60 in a lubricant to form a C60 lubricant, comprising:
combining C60 with a limonene composition to form a C60 mixture;
heating the C60 mixture to a predetermined temperature for a predetermined time period to dissolve the C60 into the limonene composition to form a C60 concentrate;
combining the C60 concentrate with a cosolvent to form a blended C60 mixture; and
combining the blended C60 mixture with the lubricant to disperse the C60 in the lubricant and to form the C60 lubricant.

2. The method as claimed in claim 1, wherein the C60 lubricant is an emulsion of the lubricant and the blended C60 mixture.

3. The method as claimed in claim 2, wherein combining the blended C60 mixture with the lubricant to form the C60 lubricant comprises:
sonicating the lubricant as the blended C60 mixture is added to the lubricant.

4. The method as claimed in claim 2, wherein combining the blended C60 mixture with the lubricant to form the C60 lubricant comprises:
mixing the lubricant with a high shear mixer as the blended C60 mixture is added to the

5. The method as claimed in claim 1, wherein:
combining the C60 concentrate with the cosolvent includes cooling the C60 concentrate to room temperature,
the C60 concentrate is at room temperature and the cosolvent is at room temperature during combining of the C60 concentrate and the cosolvent, and
the blended C60 mixture is at room temperature and the lubricant is at room temperature during combining of the blended C60 mixture and the lubricant.

6. The method as claimed in claim 1, wherein the cosolvent includes benzyl alcohol.

7. The method as claimed in claim 6, wherein the C60 concentrate and the cosolvent are mixed at a 1:1 ratio by volume.

8. The method as claimed in claim 6, wherein the C60 concentrate and the cosolvent are mixed at a 2:1 ratio by volume having two parts of the C60 concentrate and one part of the cosolvent.

9. The method as claimed in claim 1, wherein the cosolvent is miscible in limonene and is moderately polar.

10. The method as claimed in claim 1, wherein the lubricant is one of an engine oil for an internal combustion engine and a lubricating oil for a refrigerant compressor.

11. The method as claimed in claim 1, wherein the lubricant is a semi-solid lubricant.

12. The method as claimed in claim 1, wherein the C60 concentrate is homogenous and includes at least 16.67 mg of the C60 per milliliter of the limonene composition.

13. A method of forming a C60 grease, comprising:
forming the C60 lubricant of claim 1, and
combining the C60 lubricant with a thickener to form the C60 grease.

14. A method of forming a C60 lubricant, comprising:
forming a C60 mixture by combining C60 powder with a limonene composition;
heating the C60 mixture to a predetermined temperature for a predetermined time period to dissolve the C60 powder into the limonene composition to form a C60 concentrate;
forming a blended C60 mixture by combining the C60 concentrate with a cosolvent; and
forming the C60 lubricant by combining the blended C60 mixture with an oil.

15. The method as claimed in claim 14, wherein the C60 lubricant is an emulsion of the blended C60 mixture and the oil.

16. The method as claimed in claim 14, wherein the oil is a petroleum-based motor oil, a partially-synthetic motor oil, or a fully-synthetic motor oil.

17. The method as claimed in claim 14, wherein:
the oil is compatible with a refrigerant, and
the oil is used to lubricate a corresponding refrigerant compressor that compresses the refrigerant.

18. The method as claimed in claim 14, wherein the cosolvent includes benzyl alcohol.

19. The method as claimed in claim 17, wherein the C60 concentrate and the cosolvent are mixed at a 1:1 ratio by volume.

20. The method as claimed in claim 14, wherein the C60 lubricant is stable, such that no settling or precipitating of C60 powder occurs.
